(19) United States
(12) Patent Application Publication
(10) Pub. No.: US 2021/0074382 A1
(43) Pub. Date: Mar. 11, 2021

(54) SYSTEM AND METHOD FOR CATEGORIZATION OF NUCLEIC ACID SEQUENCING

(71) Applicant: KONINKLIJKE PHILIPS N.V., EINDHOVEN (NL)

(72) Inventor: Helen Cecile van Aggelen, Eindhoven (NL)

(21) Appl. No.: 16/957,441

(22) PCT Filed: Feb. 28, 2019
(86) PCT No.: PCT/EP2019/054920

§ 371 Date: Jun. 24, 2020

Related U.S. Application Data
(60) Provisional application No. 62/640,847, filed on Mar. 9, 2018.

(51) Int. Cl. G16B 30/20; C12Q 1/6869

(52) U.S. Cl. CPC G16B 30/20; C12Q 1/6869

(57) ABSTRACT

A method (100) for characterizing a genomic sample, comprising: (i) receiving (120) a first waveform from a sequencing operation for a sample, the first waveform representing a first genetic sequence; (ii) applying (130) a first function to the first waveform to generate a first waveform representation; (iii) setting (140), based on the first waveform representation, at least a first bit within a first bit array to a first value, wherein the first bit is associated with the generated first waveform representation; (iv) comparing (150) the first bit array with the first value to a second bit array, the second bit array comprising a plurality of bit values representing a set of genetic sequences; and (v) determining (160) whether the first genetic sequence is within the set of genetic sequences based on a match between the first bit array and the second bit array.

### FIELD OF THE INVENTION

The present disclosure is directed generally to methods and systems for real-time analysis and categorization of next-generation nucleic acid sequencing.

### BACKGROUND

Next-generation sequencing (NGS) is an important tool for genomics research, and has numerous applications for discovery, diagnosis, and other methodologies. For example, next-generation sequencing technologies such as nanopore sequencing make it possible to determine the composition of long nucleotide sequences by measuring changes in electric current flow through a nanopore as the nucleotide sequences move through the pore. This technology makes it possible to sequence samples in real time, and is increasingly being utilized for wide variety of applications such as diagnostics, drug resistance determination, and epidemiology, among many others.

For many applications, rapid sequencing is of upmost importance. Typical sequencing workflows for nanopore and related technologies, for example, consist of translating the output—such as the detected nanopore current changes—into k-mers, followed by analysis of the resulting sequences. Both steps can take a significant amount of computer resources and computing time. As more and more samples are characterized and stored, there is a need to harness the information and estimate or otherwise characterize the contents of samples being sequenced, such as through similarity to previously characterized samples.

### SUMMARY OF THE INVENTION

There is a continued need for rapid analysis and categorization of next-generation sequencing data to enable identification of nucleic acid in a sample.

The present disclosure is directed to inventive methods and systems for real-time analysis and categorization of next-generation nucleic acid sequencing information. Various embodiments and implementations herein are directed to a system that receives a sequencing waveform from a sequencing operation for a genomic sample. The system applies a function to the waveform to generate a waveform representation, and adjusts a bit in a first bit array to represent the waveform, and the genetic sequence that it represents, in the first bit array. The first bit array is compared to a second bit array comprising a plurality of bit values representing a plurality of genetic sequences, and the system determines whether there is a match between the two bit arrays, thereby characterizing the genomic sample. According to an embodiment, the system also receives metadata about the genomic sample, applies the first function to the metadata to generate a metadata representation, and adjusts a bit in the first bit array to represent the metadata representation.

Generally in one aspect, a method for characterizing a genomic sample. The method includes the steps of: (i) receiving a first waveform from a sequencing operation for a sample, the first waveform representing a first genetic sequence; (ii) applying a first function to the first waveform to generate a first waveform representation; (iii) setting, based on the first waveform representation, at least a first bit within a first bit array to a first value, wherein the first bit is associated with the generated first waveform representation; (iv) comparing the first bit array with the first value to a second bit array, the second bit array comprising a plurality of bit values representing a set of genetic sequences; and (v) determining whether the first genetic sequence is within the set of genetic sequences based on a match between the first bit array and the second bit array.

According to an embodiment, the method further includes: (i) receiving a second waveform from the sequencing operation for the sample, the second waveform representing a second genetic sequence; (ii) applying the first function to the second waveform to generate a second waveform representation; and (iii) setting, based on the second waveform representation, at least a second bit within the first bit array to a first value, wherein the second bit is associated with the generated second waveform representation.

According to an embodiment, the method further includes: comparing the first bit array to the second bit array; and determining whether the first genetic sequence and the second genetic sequence are within the set of genetic sequences based on a match between the first bit array and the second bit array.

According to an embodiment, the step of determining whether the first genetic sequence is within the set of genetic sequences comprises traversing a tree data structure comprising a plurality of bit arrays, each of the plurality of bit arrays representing a different subset of the set of genetic sequences.

According to an embodiment, the method further includes identifying, based on a match between the first bit array and the second bit array, the first genetic sequence.

According to an embodiment, the method further includes converting the first waveform to a first k-mer, and applying a first function to the first k-mer to generate the first waveform representation.

According to an embodiment, the first waveform is a current fluctuation.

According to an embodiment, the method further includes: receiving, with the first waveform, metadata information about the sample; applying the first function to the metadata to generate a first metadata representation; and setting, based on the first metadata representation, at least a first bit within a first bit array to a first value, wherein the first bit is associated with the first metadata representation.

According to an embodiment, the metadata comprises information about a source of the sample. According to an embodiment, the metadata comprises information about a time or date associated with the sample.

According to an embodiment, the method further includes analyzing the metadata associated with one or more genetic sequences from the sample determined to be within the set of genetic sequences.

According to an embodiment, the method further includes clustering the one or more genetic sequences from the sample determined to be within the set of genetic sequences, based at least in part on the metadata associated with the one or more genetic sequences.

According to an aspect is a system for characterizing a genomic sample. The system includes: a database a database of populated data structures each comprising one or more waveform representations each associated with known genetic sequence; a waveform module configured to: (i) apply a first function to a first waveform to generate a first waveform representation, the first waveform sequence obtained from a sequencing operation for the genomic sample and representing a first genetic sequence; and (ii) set, based on the first waveform representation, at least a first bit within a first data structure to a first value, wherein the first bit is associated with the generated first waveform representation; and a comparison module configured to: (i) compare the first data structure with the first value to one or more of the populated data structures; and (ii) determine whether the first genetic sequence is one of the known genetic sequences based on a match between the first data structure and one or more of the populated data structures.

According to an embodiment, the populated data structures are Bloom filters organized in a hierarchical tree.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for characterizing a genomic sample, in accordance with an embodiment.

FIG. 2 is a schematic representation of sequencing waveforms, in accordance with an embodiment.

FIG. 3 is a schematic representation of a function applied to a sequencing waveform, in accordance with an embodiment.

FIG. 4 is a schematic representation of a data structure comprising one or more sequencing waveform representations, in accordance with an embodiment.

FIG. 5 is a schematic representation of a hierarchical data structure, in accordance with an embodiment.

FIG. 6 is a schematic representation of data structures comprising one or more sequencing waveform representations and one or more metadata representations, in accordance with an embodiment.

FIG. 7 is a schematic representation of a sequence characterization system, in accordance with an embodiment.

### DETAILED DESCRIPTION OF EMBODIMENTS

The present disclosure describes various embodiments of a system and method for characterizing a genomic sample using waveforms generated by next-generation sequencing platforms. More generally, Applicant has recognized and appreciated that it would be beneficial to provide a system that enables rapid identification of nucleic acids within a genomic sample. The system, which may optionally comprise a sequencer, receives a sequencing waveform from a sequencing operation for the sample and/or retrieves a stored sequencing waveform. The sequencing waveform, which may be the measurement of an electrical current across a pore among many other waveforms, represents a nucleic acid sequence. The system applies a function or operation to the waveform to generate a waveform representation, and then adjusts one or more bits in a first bit array such that the first bit array now includes the waveform representation. To characterize the nucleic acid sequence, the system compares the first bit array to a second bit array comprising a plurality of bit values representing a plurality of genetic sequences, and determines whether there is a match between the two bit arrays. If there is a match, then the nucleic acid represented by the waveform is partially or wholly characterized or identified.

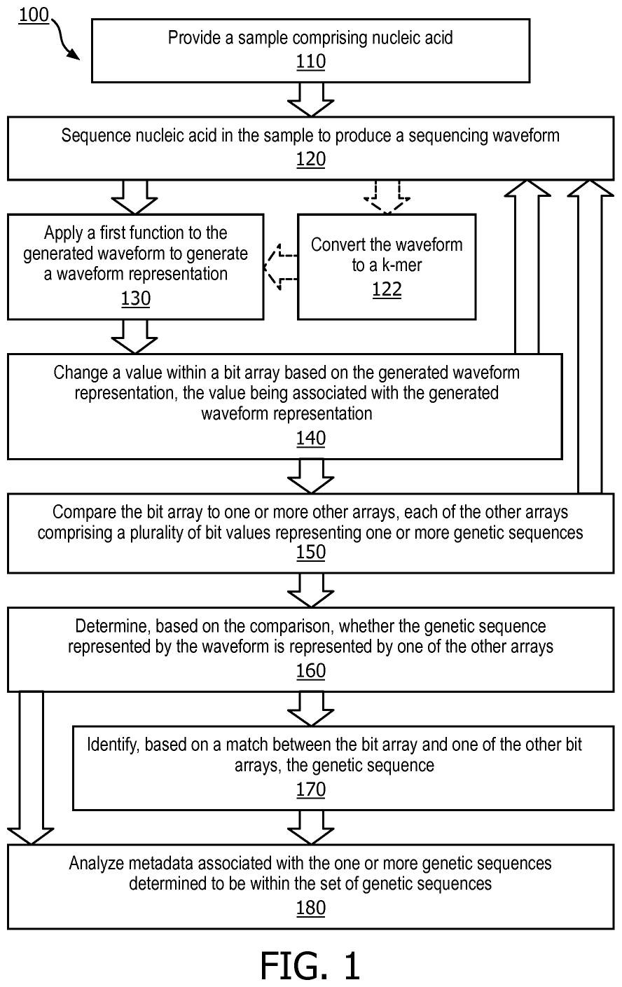

Referring to FIG. 1, in one embodiment, is a flowchart of a method 100 for characterizing a genomic sample using waveforms generated by next-generation sequencing platforms. At step 110 of the method, a sample comprising or potentially comprising nucleic acid to be sequenced is provided or received. The sample may comprise nucleic acid from one or more microorganisms such as bacteria, viruses, fungi, and/or from plants or animals, among many other sources. A sample may comprise nucleic acid molecules from one organism or from multiple organisms. Samples may be obtained in a clinical setting, from the environment, from indoor or outdoor surfaces, or from any other source. It is recognized that there is no limitation to the source of the sample, or the nucleic acid(s) in the sample.

The sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner.

At step 120 of the method, the sequencing platform sequences at least a portion of a nucleic acid from the sample, thereby generating a sequencing waveform in real time. The sequencing waveform represents the sequence of the nucleic acid being sequenced, and can be any waveform representative of a genetic sequence. The sequencing platform can be any sequencing platform, including but not limited to any systems described or otherwise envisioned herein. For example, the sequencing platform can be a real-time single-molecule sequencing platform, such as a pore-based sequencing platform, although many other sequencing platforms are possible.

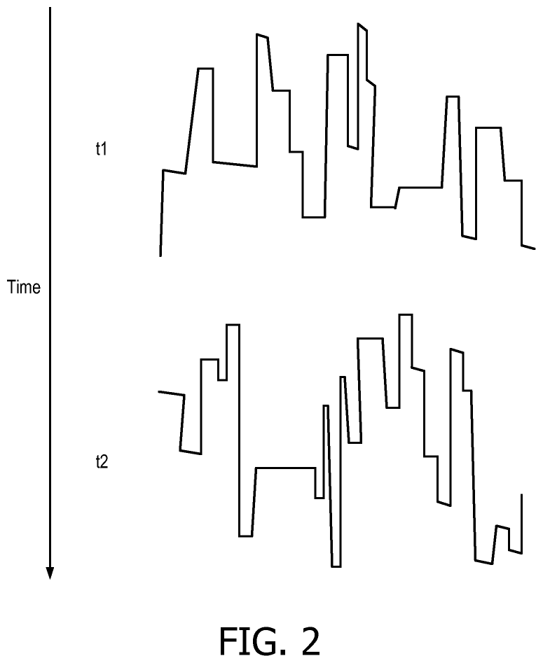

According to an embodiment, the sequencing platform is a pore-based sequencing platform. As a single nucleic acid strand passes through the pore, the bases affect a current flow through the pore as detected by a current meter. Each type of base (A, C, G, and T) has a slightly different effect on the current flow through the pore, and thus the waveform generated by the changing current flow is representative of the sequence of nucleic acid bases that pass through the pore. An example of two waveforms, t1 and t2, is provided in FIG. 2, which is an approximation or estimate of a shape and/or magnitude of expected current flow signal through the pore generated by the presence of an A, C, G, or T base. In many systems the generated waveform is interpreted to reveal the underlying genetic sequence of the nucleic acid strand that passed through the pore.

According to an embodiment, the sequencing waveform is communicated to or from the sequencing platform to a controller or other analysis module for downstream analysis and characterization such as identification of the nucleic acid sequence and/or the sample. For example, according to one embodiment the sequencing platform may comprise a controller or other analysis module for downstream analysis and characterization. According to another embodiment, the sequencing platform communicates the generated sequencing waveform, in real-time or at certain time points, to a local or remote controller or other analysis module for downstream analysis and characterization.

At optional step 122 of the method, the generated waveform is converted to a k-mer that represents the underlying genetic sequence of the nucleic acid strand that passed through the pore. For example, the system may comprise a controller or module configured or programmed to convert the waveform to a k-mer using known methods for conversion.

At step 130 of the method, a first function is applied to the generated waveform to generate a first waveform representation. Alternatively, the first function is applied to the k-mer resulting from interpretation of the waveform. The function can be applied to the waveform in real-time as it is generated, or can be applied at any point during or after sequencing. The first function can be any function that generates a waveform representation. According to an embodiment, the function converts a waveform of arbitrary size to a data point of fixed size. A hash function, for example, can convert a waveform of arbitrary size to a hash value of fixed size, typically comprising one or more integers. The fixed size can be any size sufficient for, for example, the system to represent the variety of genetic sequences for which the system is designed or programmed.

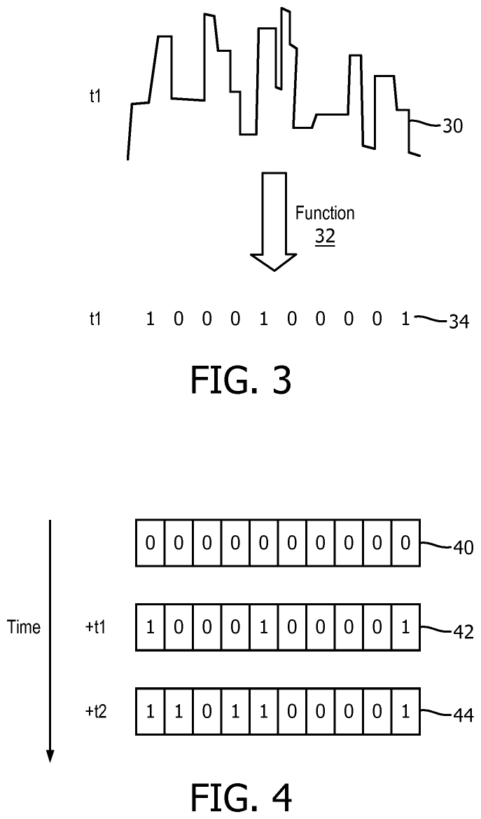

For example, referring to FIG. 3 is a schematic representation of a function 32 applied to a generated waveform 30 to generate a first waveform representation 34. The function can be a hash function configured to generate one or more bits for a bit array, as shown in FIG. 3, although many other functions are possible.

At step 140 of the method, one or more bits within a bit array are set to a new value based on the generated waveform representation from the first function. The one or more bit values are associated with the generated waveform representation. For example, referring to FIG. 4 is a schematic representation of two generated waveform representations, t1 and t2, being added to a bit array 40. According to an embodiment, bit array 40 is a Bloom filter. Initially the bit array 40 will comprise no waveform representations. When t1 is added to bit array 40, one or more bits in bit array 40 are changed. In this example, one or more bits are changed from “0” to “1” to represent the waveform representation 34 (i.e., t1). Accordingly, the new bit array 42 comprises waveform representation 34. When t2 is added to bit array 42, one or more bits in bit array 42 are changed from “0” to “1” to represent the waveform representation for t2. Accordingly, the new bit array 44 comprises both waveform representations t1 and t2. As the sequencing continues and new waveform representations representing k-mers or waveforms are detected, more bits in the bit array will be changed. Notably, the function can be performed and the waveform representation can be integrated into the bit array in real-time as the sequencer generates a waveform.

According to one embodiment, the system can monitor the progress of a sequencing analysis. For example, by monitoring the rate that new values in the bit array are changed, it is possible to estimate whether the sequencing process is reaching a saturation point. If values are frequently changed in the bit array as waveform representations are added, new genetic sequences are being obtained. If waveform representations are added to the bit array without a change it bit values, then repetitive genetic sequences are being obtained. A timer or other timing function can be implemented to obtain a rate of new genetic sequences being added to the bit array, and a monitor can characterize the sequencing process, such as determining whether sequencing should be terminated, based on the timing function and/or other aspects of changes to the bit array.

According to an embodiment, the system changes the one or more bits within the bit array based on the generated waveform representation only if a threshold number of first waveform representations are generated or counted. For example, the system may comprise a counter that counts the number of a specific waveform representation that is generated, which represents a number of times that a specific genetic sequence is sequenced or obtained by the system. This may be utilized to minimize false positive identification of sequences by requiring the system to identify the genetic sequence a certain number of times before it is added to the bit array.

According to an embodiment, the system returns to step 120 to receive a second waveform from the sequencing operation for the sample, the second waveform representing a second genetic sequence. Alternatively, the system returns to step 120 to retrieve a second waveform from a database of stored waveforms. The system will apply the first function to the second waveform to generate a second waveform representation at step 130 of the method, and can set, based on the second waveform representation, one or more bits within the bit array to a new value. In this way, the bit array can accumulate any number of genetic sequences, from one to many sequences. The system can be programmed, designed, or otherwise controlled to obtain a certain number or quantity of sequences, ranging from one to two or more.

At step 150 of the method, the system compares the bit array containing one or more waveform representations to one or more other bit arrays, each of the other bit arrays comprising a plurality of bit values representing one or more genetic sequences. Each bit array can comprise a single genetic sequence or a set of two or more genetic sequences. This comparison can be accomplished via any known method for bit comparison. The system can be programmed to require an exact match between the bit array containing the waveform representation(s) and another bit array, or a close match between the arrays. The quality of the match can be a setting selected by a user or otherwise programmed into the system.

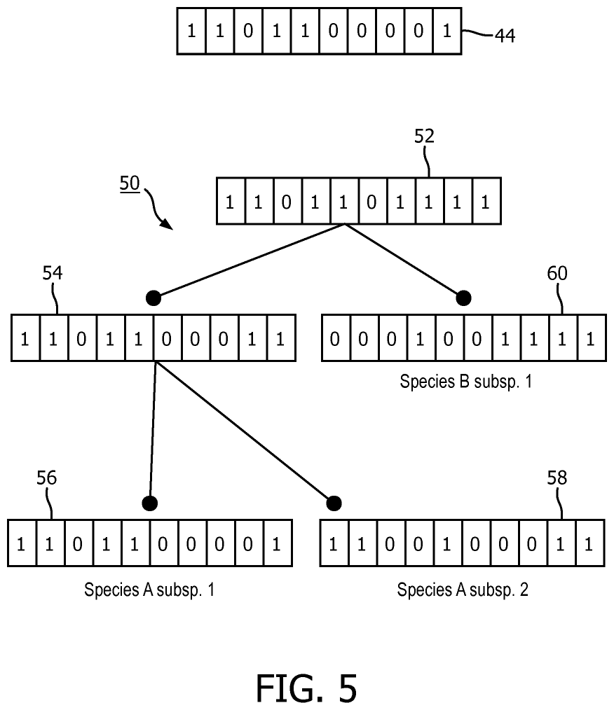

Referring to FIG. 5, in one embodiment, is a schematic representation of a method or system for comparing bit arrays. In this system, the other bit arrays comprise a hierarchical tree structure 50, where the tree data structure comprises at least a root node and a plurality of leaf nodes. According to an embodiment, the bit arrays in the hierarchical tree structure 50 are Bloom filters, each Bloom filter representing one or more previously characterized samples or previously sequenced genetic data. However, many other data structures are possible.

Typically, in a hierarchical tree structure such as that shown in FIG. 5, a bit value representing waveform will be inserted into the tree from the bottom up. Thus, bit array 56 contains just data for Species A, subspecies 1, which can be one genetic sequence or a set of genetic sequences. Similarly, bit array 58 contains just data for Species A, subspecies 2, which can be one genetic sequence or a set of genetic sequences. However, bit array 54 will contain data for both Species A, subspecies 1 and Species A, subspecies 2. Similarly, bit array 52 will contain data for Species A, subspecies 1, Species A, subspecies 2, and Species B, subspecies 1. Thus, the hierarchical tree structure can be traversed from the top down to identify the one genetic sequence or set of genetic sequences within the queried bit array 44.

At step 160 of the method, the system determines from the comparison whether a genetic sequence represented by the waveform representation in the first bit array is within a set of one or more genetic sequences represented by a second bit array. This is accomplished, for example, by looking for a match of values between the first bit array containing the waveform representation and values within another bit array. For example, referring to FIG. 5, bit array 44 is compared to bit array 52. If the data contained within bit array 44 is also contained with bit array 44, the system will progress to the next branch of the tree. Bit array 44 will then be compared to both bit array 54 and bit array 60 to determine whether the data contained with bit array 44 is contained within either. Since the waveform representation found within bit array 44 is found within bit array 54 but not bit array 60, the system will compare bit array 44 to the next branch of the tree, namely bit arrays 56 and 58. In this example, the waveform representations (t1 and t2) found within bit array 44 are found within bit array 56, and thus bit array 44 is characterized or identified as comprising or otherwise related to Species A, subspecies 1, which can represent one or more genetic sequences and/or other information. Bit array 56 may contain only the genetic sequences contained within bit array 44, or bit array 56 may contain more than the genetic sequences contained within bit array 44. There is no limit on the number of arrays that can be included within the hierarchical tree structure. The hierarchical tree structure can be a binary tree, an AVL tree, a B+ tree, or a wide variety of other trees. Additionally, rather than a Bloom filter, the data structures can be a counting Bloom filter, and the filter can be compressed.

At optional step 170 of the method, the system identifies the genetic sequence or sequences represented by the bit array generated from sequencing, based on the determined match between the bit array containing the waveform representation and the known matching bit array. According to an embodiment, and referring again to FIG. 5, finding a match between bit array 44 and bit array 56 is sufficient to characterize the sample from which bit array 44 was generated. However, according to another embodiment, the match between bit array 44 and bit array 56 identifies with greater specificity the genetic sequence or sequences within bit array 44. This can be determined by the needs of the system. In some embodiments, a match or sufficient similarity between bit array 44 and bit array 56 can be enough to be diagnostic or otherwise informative for some purposes. In other embodiments, matching between bit array 44 and bit array 56 reveals the exact set of genetic sequences contained within bit array 44, which may be required for some diagnostic or other purposes.

At optional step 180 of the method, the system analyzes metadata associated with the genetic sequences from the sample determined to be within the set of genetic sequences, based on matching between the bit array containing the waveform representation and the known matching bit array.

According to an embodiment, the data structure comprises metadata associated with the sample or genetic sequence(s) within the sample. Accordingly, at step 120 of the method, the system receives, together with the sample and/or the waveform generated from a nucleic acid strand in the sample, metadata about the sample. At step 130 of the method, the first function is applied to the metadata to generate a metadata representation. At step 140, one or more bits within the bit array are set to a new value based on the generated metadata representation from the first function. A portion of the bit vector can be reserved to encode metadata, such as a time and/or location stamp. For example, the bit vector can comprise 365 bits to encode the days a patient spent in a hospital, and/or 10 bits to encode a ward number.

Thus, the bit array utilized in steps 150, 160, and 170 of the method will comprise not only bits for the waveform representation, but also bits for the metadata representation. The metadata can be any information about or otherwise associated with the sample. For example, the metadata can be a location of the sample, a time or date of the sample, patient information, and/or any other information.

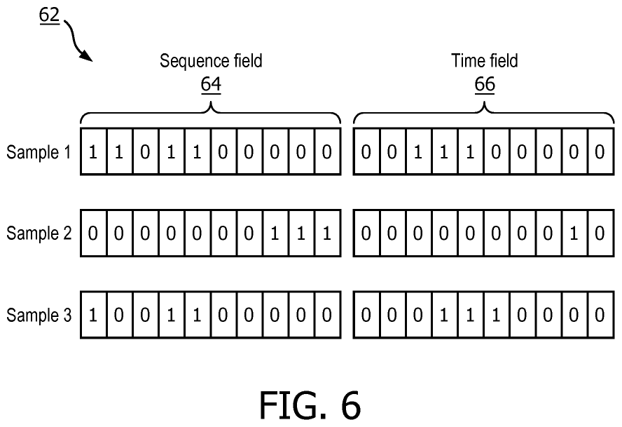

Referring to FIG. 6, in one embodiment, is a series of bit arrays for a series of samples (sample 1, sample 2, and sample 3). Each bit array generated by the methods described or otherwise envisioned herein comprises information about the waveform representation encoded within the sequence field 64, and information about the metadata representation encoded within the time field 66. Although called “time field” in FIG. 6, it is understood that the field may not encode time, and may encode any information associated with the genetic sequence, sample, or waveform representation. According to an embodiment, the time field 66 is a counting Bloom filter in which taking the union of filters increases the count of overlapping bits. Accordingly, a histogram for each branch of the hierarchical tree structure can be visualized to reveal peak times, peak locations, or any other metadata information.

At step 150 of the method, the system compares one or more bit arrays containing one or more waveform representations to one or more other bit arrays, each of the other bit arrays comprising a plurality of bit values representing one or more genetic sequences. The metadata can optionally be ignored until a match is found between the queried bit array and one of the known bit arrays, such as a bit array within the hierarchical tree structure. Once a bit array is characterized with regard to the waveform representation(s) it contains, the metadata associated with those waveform representations can be analyzed. This may, for example, cluster together metadata based on similarity of genetic sequences, which allows for analysis of the clustering metadata. According to just one example in a clinical setting, sequencing of many different samples within a hospital setting may identify a pathogen in a number of samples using the methods described herein. The metadata associated with the samples within which the pathogen is identified can be analyzed to determine the source of the sample, the date/time the sample was obtained, a possible route or vector for the pathogen, and many other aspects. Many other clinical and non-clinical examples are possible. According to an embodiment, therefore, step 170 of the method comprises clustering the one or more genetic sequences from the sample determined to be within the set of genetic sequences, based at least in part on the metadata associated with the one or more genetic sequences.

According to another embodiment, at step 150 of the method, the system can compare one or more bit arrays containing one or more metadata representations to one or more other bit arrays, each of the other bit arrays comprising one or more bit values representing metadata. In this embodiment, the waveform representations can optionally be ignored until a match is found between the queried bit array and one of the known bit arrays, such as a bit array within the hierarchical tree structure. Once a bit array is characterized with regard to the metadata representation(s) it contains, the waveforms associated with those metadata representations can be analyzed. This may, for example, cluster together genetic sequences based on similarity of metadata, which allows for analysis of the clustering genetic sequences. According to just one example in a clinical setting, a particular location may be swabbed for sequencing on a routine basis, and the location and/or date and time of the swabbing can be encoded in bit arrays. The genetic sequences that are identified based on matching via metadata representations can then be analyzed.

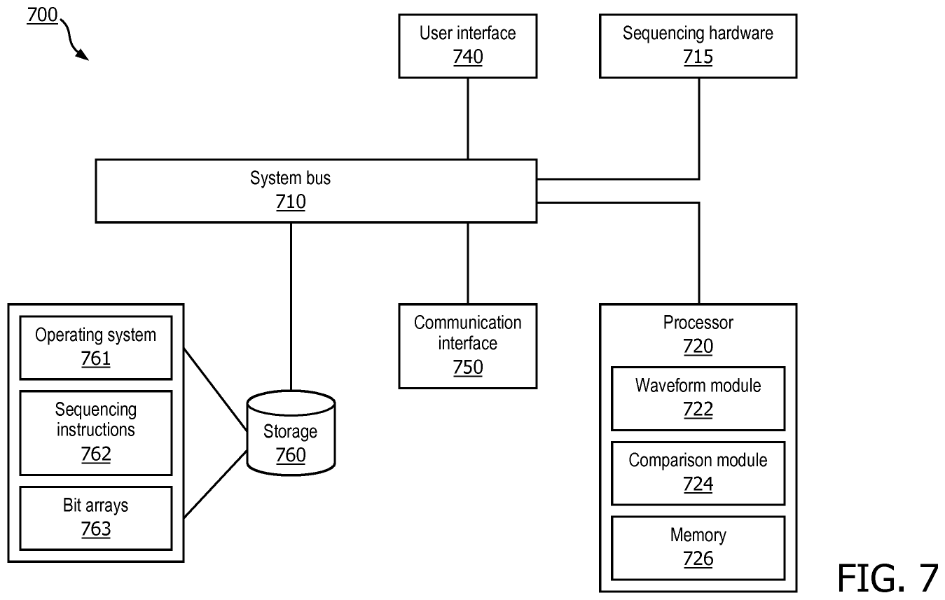

Referring to FIG. 7, in one embodiment, is a schematic representation of a system 700 for characterizing a genomic sample using waveforms generated by next-generation sequencing platforms. System 700 includes one or more of a processor 720, memory 726, user interface 740, communications interface 750, and storage 760, interconnected via one or more system buses 710. In some embodiments, such as those where the system comprises or implements a sequencer or sequencing platform, the hardware may include additional sequencing hardware 715 such as a real-time single-molecule sequencer, including but not limited to a pore-based sequencer, although many other sequencing platforms are possible. It will be understood that FIG. 7 constitutes, in some respects, an abstraction and that the actual organization of the components of the system 700 may be different and more complex than illustrated.

According to an embodiment, system 700 comprises a processor 720 capable of executing instructions stored in memory 726 or storage 760 or otherwise processing data. Processor 720 performs one or more steps of the method, and may comprise one or more of the modules described or otherwise envisioned herein. Processor 720 may be formed of one or multiple modules, and can comprise, for example, a memory 726. Processor 720 may take any suitable form, including but not limited to a microprocessor, microcontroller, multiple microcontrollers, circuitry, field programmable gate array (FPGA), application-specific integrated circuit (ASIC), a single processor, or plural processors.

Memory 726 can take any suitable form, including a non-volatile memory and/or RAM. The memory 726 may include various memories such as, for example L1, L2, or L3 cache or system memory. As such, the memory 726 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 700. It will be apparent that, in embodiments where the processor implements one or more of the functions described herein in hardware, the software described as corresponding to such functionality in other embodiments may be omitted.

User interface 740 may include one or more devices for enabling communication with a user such as an administrator. The user interface can be any device or system that allows information to be conveyed and/or received, and may include a display, a mouse, and/or a keyboard for receiving user commands. In some embodiments, user interface 740 may include a command line interface or graphical user interface that may be presented to a remote terminal via communication interface 750. The user interface may be located with one or more other components of the system, or may located remote from the system and in communication via a wired and/or wireless communications network.

Communication interface 750 may include one or more devices for enabling communication with other hardware devices. For example, communication interface 2750 may include a network interface card (NIC) configured to communicate according to the Ethernet protocol. Additionally, communication interface 750 may implement a TCP/IP stack for communication according to the TCP/IP protocols. Various alternative or additional hardware or configurations for communication interface 750 will be apparent.

Storage 760 may include one or more machine-readable storage media such as read-only memory (ROM), random-access memory (RAM), magnetic disk storage media, optical storage media, flash-memory devices, or similar storage media. In various embodiments, storage 760 may store instructions for execution by processor 720 or data upon which processor 720 may operate. For example, storage 760 may store an operating system 761 for controlling various operations of system 700. Where system 700 implements a sequencer and includes sequencing hardware 715, storage 760 may include sequencing instructions 762 for operating the sequencing hardware 715. Storage 760 may also store one or more bit arrays 763 used by the system to identify or otherwise characterize genetic sequences.

It will be apparent that various information described as stored in storage 760 may be additionally or alternatively stored in memory 726. In this respect, memory 726 may also be considered to constitute a storage device and storage 760 may be considered a memory. Various other arrangements will be apparent. Further, memory 726 and storage 760 may both be considered to be non-transitory machine-readable media. As used herein, the term non-transitory will be understood to exclude transitory signals but to include all forms of storage, including both volatile and non-volatile memories.

While system 700 is shown as including one of each described component, the various components may be duplicated in various embodiments. For example, processor 720 may include multiple microprocessors that are configured to independently execute the methods described herein or are configured to perform steps or subroutines of the methods described herein such that the multiple processors cooperate to achieve the functionality described herein. Further, where system 700 is implemented in a cloud computing system, the various hardware components may belong to separate physical systems. For example, processor 720 may include a first processor in a first server and a second processor in a second server. Many other variations and configurations are possible.

According to an embodiment, processor 720 comprises one or more modules to carry out one or more functions or steps of the methods described or otherwise envisioned herein. For example, processor 720 may comprise a waveform module 722 and/or a comparison module 724. According to an embodiment, waveform module 722 receives a waveform generated by a sequencing platform such as sequencing hardware 715. The waveform module 722 applies the first function to the generated waveform to generate a first waveform representation. Waveform module 722 may optionally apply the first function to a k-mer resulting from interpretation of the waveform. The function can be applied to the waveform in real-time as it is generated, or can be applied at any point during or after sequencing. The first function can be any function that generates a waveform representation. According to an embodiment, the function converts a waveform of arbitrary size to a data point of fixed size. A hash function, for example, can convert a waveform of arbitrary size to a hash value of fixed size, typically comprising one or more integers. The fixed size can be any size sufficient for, for example, the system to represent the variety of genetic sequences for which the system is designed or programmed. According to an embodiment, waveform module 722 applies the first function to metadata received by the system to generate a metadata representation. Waveform module 722 also generates a new bit array or modifies an existing bit array with the data from the waveform representation and/or the metadata representation. For example, according to an embodiment, one or more bits within a bit array are set to a new value based on the generated waveform representation and/or metadata representation from the first function.

According to an embodiment, processor 720 comprises a comparison module 724. According to an embodiment, comparison module 724 compares the bit array containing one or more waveform representations to one or more other bit arrays, each of the other bit arrays comprising a plurality of bit values representing one or more genetic sequences. The other bit arrays can be, for example, bit arrays 763 in storage 760, among other possibilities. This comparison can be accomplished via any known method for bit comparison. The comparison can be performed, for example, via a hierarchical tree structure as described or otherwise envisioned herein. The comparison module 724 determines from the comparison whether a genetic sequence represented by the waveform representation in the first bit array is within a set of one or more genetic sequences represented by a second bit array. The comparison module 724 may then identify the genetic sequence or sequences represented by the bit array based on the determined match between the bit array containing the waveform representation and the known matching bit array. Optionally, the comparison module 724 analyzes metadata associated with the genetic sequences from the sample determined to be within the set of genetic sequences, based on matching between the bit array containing the waveform representation and the known matching bit array or arrays.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for characterizing a genomic sample, comprising:
receiving a first waveform from a sequencing operation for a sample, the first waveform representing a first genetic sequence;
applying a first function to the first waveform to generate a first waveform representation;
setting, based on the first waveform representation, at least a first bit within a first bit array to a first value, wherein the first bit is associated with the generated first waveform representation;
comparing the first bit array with the first value to a second bit array, the second bit array comprising a plurality of bit values representing a set of genetic sequences; and
determining whether the first genetic sequence is within the set of genetic sequences based on a match between the first bit array and the second bit array.

2. The method of claim 1, further comprising:
receiving a second waveform from the sequencing operation for the sample, the second waveform representing a second genetic sequence;
applying the first function to the second waveform to generate a second waveform representation; and
setting, based on the second waveform representation, at least a second bit within the first bit array to a first value, wherein the second bit is associated with the generated second waveform representation.

3. The method of claim 2, further comprising the steps of:
comparing the first bit array to the second bit array; and
determining whether the first genetic sequence and the second genetic sequence are within the set of genetic sequences based on a match between the first bit array and the second bit array.

4. The method of claim 1, wherein the step of determining whether the first genetic sequence is within the set of genetic sequences comprises traversing a tree data structure comprising a plurality of bit arrays, each of the plurality of bit arrays representing a different subset of the set of genetic sequences.

5. The method of claim 1, further comprising the step of identifying, based on a match between the first bit array and the second bit array, the first genetic sequence.

6. The method of claim 1, further comprising the step of converting the first waveform to a first k-mer, and applying a first function to the first k-mer to generate the first waveform representation.

7. The method of claim 1, wherein the first waveform is a current fluctuation.

8. The method of claim 1, further comprising:
receiving, with the first waveform, metadata information about the sample;
applying the first function to the metadata to generate a first metadata representation; and
setting, based on the first metadata representation, at least a first bit within a first bit array to a first value, wherein the first bit is associated with the first metadata representation.

9. The method of claim 8, wherein the metadata comprises information about a source of the sample.

10. The method of claim 8, wherein the metadata comprises information about a time or date associated with the sample.

11. The method of claim 8, further comprising the step of analyzing the metadata associated with one or more genetic sequences from the sample determined to be within the set of genetic sequences.

12. The method of claim 8, further comprising the step of clustering the one or more genetic sequences from the sample determined to be within the set of genetic sequences, based at least in part on the metadata associated with the one or more genetic sequences.

13. A system for characterizing a genomic sample, comprising:
a database of populated data structures each comprising one or more waveform representations each associated with known genetic sequence;
a waveform module configured to: (i) apply a first function to a first waveform to generate a first waveform representation, the first waveform sequence obtained from a sequencing operation for the genomic sample and representing a first genetic sequence; and (ii) set, based on the first waveform representation, at least a first bit within a first data structure to a first value, wherein the first bit is associated with the generated first waveform representation; and
a comparison module configured to: (i) compare the first data structure with the first value to one or more of the populated data structures; and (ii) determine whether the first genetic sequence is one of the known genetic sequences based on a match between the first data structure and one or more of the populated data structures.

14. The system of claim 13, wherein the first waveform is a current fluctuation.

15. The system of claim 13, wherein the populated data structures are Bloom filters organized in a hierarchical tree.
